(19) United States
(12) Patent Application Publication
(10) Pub. No.: US 2026/0060669 A1
(43) Pub. Date: Mar. 5, 2026

(54) SURGICAL ANCHOR INSERTER WITH STRAND SECURING DEVICE

(71) Applicant: Arthrex, Inc., Naples, FL (US)

(72) Inventors: Peter J. DREYFUSS, Naples, FL (US); Jeffrey R. DUGAS, Vestavia Hills, AL (US); E. Lyle CAIN, JR., Birmingham, AL (US)

(21) Appl. No.: 19/318,819

(22) Filed: Sep. 4, 2025

Related U.S. Application Data
(60) Provisional application No. 63/690,659, filed on Sep. 4, 2024.

(51) Int. Cl. A61B 17/04; A61B 17/00; A61F 2/08

(52) U.S. Cl. CPC A61B 17/0401; A61F 2/0811; A61B 2017/00367; A61B 2017/0409; A61B 2017/0464; A61F 2002/0852; A61F 2002/0888

(57) ABSTRACT

An inserter for tissue repair includes a shaft that operatively engages an implant. The implant is coupled to at least one flexible strand. A strand securing device is located at the proximal end of the shaft. The strand securing device has a base portion and a handle portion moveably coupled to the base portion between a first position and a second position.

### CROSS-REFERENCE TO RELATED APPLICATION

This application claims the benefit of U.S. Provisional Patent App. No. 63/690,659, filed Sep. 4, 2024, the disclosure of which is hereby incorporated by reference herein

### TECHNICAL FIELD

The disclosure relates generally to surgical anchor inserters used to repair tissue.

### BACKGROUND

When soft tissue is torn or becomes detached from bone, surgery is usually required to reattach or reconstruct the tissue. Inserters are commonly used in such surgical procedures to insert, deploy, and/or or position anchors or implants to be used in repairing the tissue. Use of the inserter, including management of the sutures used for the repair, can be cumbersome and can contribute to surgical errors.

### SUMMARY

The present disclosure generally provides an inserter for tissue repair that comprises a shaft configured to operatively engage an implant which may be coupled to at least one flexible strand, and a strand securing device at the end of the shaft.

The shaft may have a proximal end and a distal end. The strand securing device may be located at the proximal end of the shaft. The strand securing device may have a base portion and a handle portion moveably coupled to the base portion between a first position and a second position. When the handle portion is in the first position, the base portion may be configured to receive free ends of the at least one flexible strand, and when the handle portion is in the second position, the free ends of the at least one flexible strand may be retained between the base portion and the handle portion.

In some aspects of the present disclosure, the handle portion may be pivotably coupled to the base portion such that the handle portion pivots between the first and second positions with respect to the base portion; the base portion may comprise a receiving area configured to receive the free ends of the at least one flexible strand, such that the free ends of the at least one flexible strand are wrapped around the base portion; the receiving area may be an annular groove extending substantially around the base portion; the strand securing device may be located at a most proximal end of the inserter; and/or when the handle portion is in the first position, the handle portion may extend in a substantially longitudinal direction with respect to a longitudinal axis of the shaft, and when the handle portion is in the second position, the handle portion may extend in a substantially transverse direction with respect to the longitudinal axis of the shaft.

In other aspects, the base portion may include a base engagement feature configured to receive the free ends of the at least one flexible strand; the handle portion may include a handle engagement feature configured to releasably engage the base engagement feature when the handle portion is in the second position; the base engagement feature may be a longitudinal slot and the handle engagement feature may be a projection extending from the handle portion; the base engagement feature and the handle engagement feature may frictionally engage; the handle portion may include a gripping feature configured to facilitate gripping of the handle portion; the gripping feature may comprise a half-ring that defines an open area for gripping the half-ring; the handle portion may include an actuator for moving the handle portion between the first and second positions; the actuator may be a tab extending outwardly from the handle portion; the base portion may include at least one flange configured to support the handle portion when the handle portion is in the second position; the base portion comprises a receiving area configured to receive the free ends of the at least one flexible strand such that the free ends of the at least one flexible strand are wrapped around the base portion; and/or the receiving area is an annular groove extending substantially around the base portion.

The present disclosure may also provide an inserter for tissue repair that comprises a shaft that may have a proximal end and a distal end, the distal end may be configured to operatively engage an implant and the implant may be coupled to at least one flexible strand. A strand securing device may be located at the proximal end of the shaft. The strand securing device may have a base portion and a handle portion moveably coupled to the base portion between a first position and a second position. The base portion may comprise a receiving area for receiving free ends of the at least one flexible strand. The base portion may have a base engagement feature and the handle portion may have a handle engagement feature to releasably retain the free ends of the at least one flexible strand between the base portion and the handle portion when the handle portion is in the second position.

In certain aspects, the base portion may include at least one flange configured to support the handle portion when the handle portion is in the second position; the base and handle engagement features may engage in a frictional engagement when the handle portion is in the second position; the handle portion may include a gripping feature configured to facilitate gripping of the handle portion; the handle portion may include an actuator for moving the handle portion between the first and second positions; the handle portion may be pivotably coupled to the base portion such that the handle portion pivots between the first and second positions with respect to the base portion; and/or the receiving area is an annular groove extending substantially around the base portion.

This summary is not intended to identify essential features of the claimed subject matter, nor is it intended for use in determining the scope of the claimed subject matter. It is to be understood that both the foregoing general description and the following detailed description are exemplary and are intended to provide an overview or framework to understand the nature and character of the disclosure.

### BRIEF DESCRIPTION OF THE FIGURES

The accompanying drawings are incorporated in and constitute a part of this specification. It is to be understood that the drawings illustrate only some examples of the disclosure and other examples or combinations of various examples that are not specifically illustrated in the figures may still fall within the scope of this disclosure. Examples will now be described with additional detail through the use of the drawings, in which:

FIG. 1 is a perspective view of an exemplary inserter;

FIG. 2 is an enlarged partial elevational view of the inserter illustrated in FIG. 1, showing a strand securing device of the inserter and flexible strands retained by the strand securing device;

FIG. 3 is another elevational view of the inserter illustrated in FIG. 1, showing the strand securing device retaining flexible strands;

FIG. 4A is a partial perspective view of the inserter, similar to FIG. 2, showing a handle portion of the strand securing device in a first position; and

FIG. 4B is another perspective view of the inserter, similar to FIG. 2, showing the handle portion of the strand securing device in a second position.

### DETAILED DESCRIPTION

It should be understood that any one of the described features may be used separately or in combination with other features. Other invented devices, systems, methods, features, and advantages will be or become apparent to one with skill in the art upon examining the drawings and the detailed description herein. It is intended that all such additional devices, systems, methods, features, and advantages be protected by the accompanying claims.

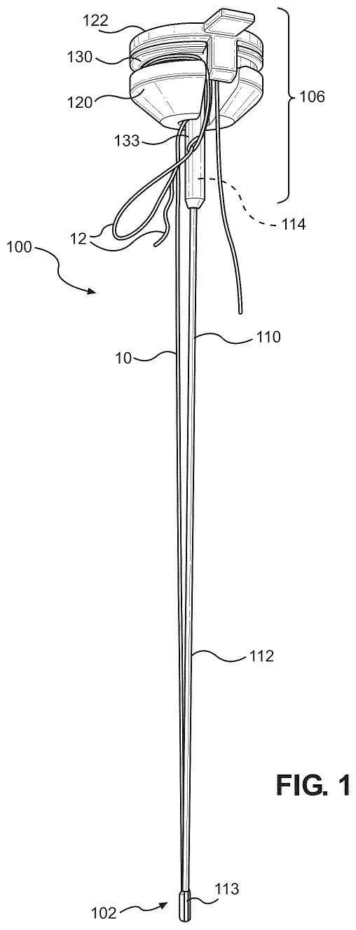

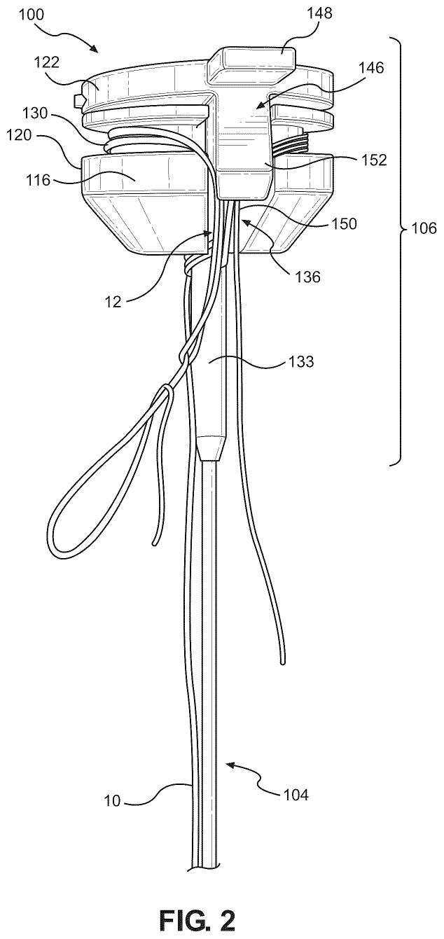

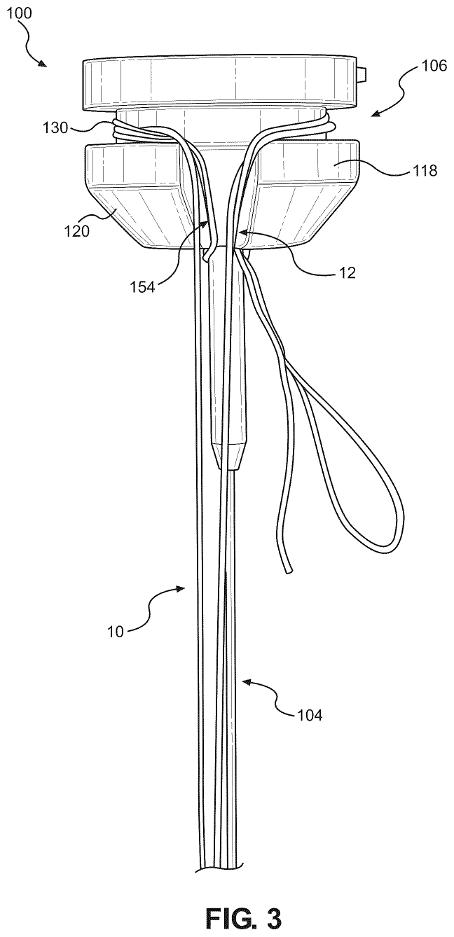

Referring to the figures, the present disclosure generally provides an inserter for tissue repair that provides ease of both use and handling as well as management of flexible strands used for the repair. FIG. 1 illustrates an exemplary inserter 100 according to the present disclosure, showing the inserter 100 with an implant 102 and one or more flexible strands 10 loaded onto inserter 100. FIGS. 2 and 3 are opposite elevational views, respectively, of inserter 100, showing free ends 12 of the flexible strands 10 neatly retained by inserter 100. Inserter 100 may be configured for use in any surgical tissue repair procedure in which soft tissue is being reattached or reconstructed. Inserter 100 generally comprises a shaft 104 configured to operatively engage implant 102 and a strand securing device 106 for managing the free ends 12 of the flexible strands 10. The one or more flexible strands 10 may be any type of flexible material used in connection with the tissue repair, e.g. repair or retention strands, such as suture, suture tape, or the like. Implant 102 can be any tissue repair implant or fixation device, such as an all suture soft implant, threaded suture anchors, or the like. The flexible strands 10 can be coupled to implant 102, as seen in FIG. 1, such as by threading or passing the flexible strands 10 through or around implant 102.

Shaft 104 may be elongated and may have a proximal end 110 and a distal end 112, as seen in FIG. 1. Distal end 112 can be configured to operatively engage implant 102. For example, distal end 112 may incorporate a tip 113 for engaging implant 102, such as a notch, fork, eyelet, recess, or the like. The free ends 12 of the flexible strands 10 are coupled to strand securing device 106 to retain implant 102 at distal end 112 such that implant 102 is in a ready position for installation or positioning in connection with the tissue repair procedure. Inserter 100 can be used to anchor implant 102, such as by installing implant 102 into a bone hole or positioning implant 102 on a tissue surface.

Strand securing device 106 can be positioned at the proximal end 110 of shaft 104. In one example, strand securing device 106 may be located at a most proximal end 114 of shaft 104 for ease of access by the surgeon. Strand securing device 106 may be configured to facilitate both the surgeon's use of inserter 100, such as a handle for the surgeon, and the management of the flexible strands 10 during a tissue repair procedure. As seen in FIGS. 2 and 3, strand securing device 106 retains and manages the free ends 12 of the flexible strands 10 making it easier for the surgeon to retain, organize, and distinguish the various flexible strands 10. For example, the free ends 12 of the flexible strands 10 can be wrapped around strand securing device 106 with the flexible strands extending along one or more sides 116 and 118 (FIG. 2 showing side 116 and FIG. 3 showing side 118) of strand securing device 106.

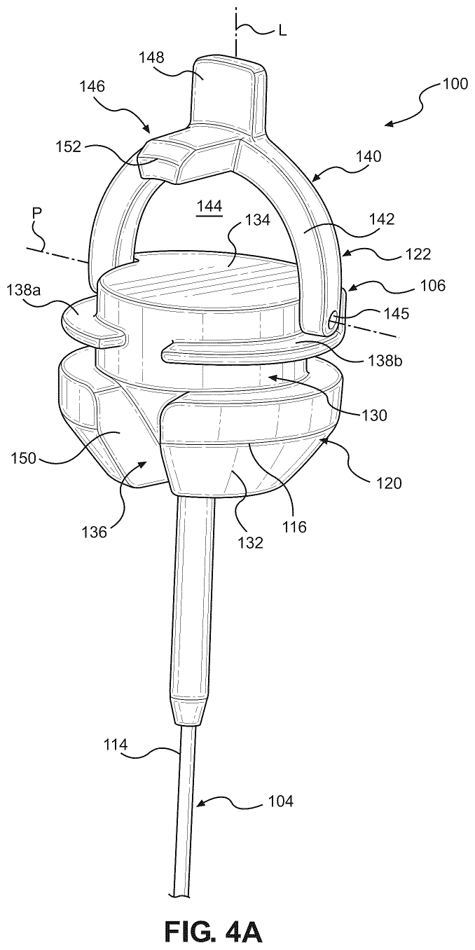

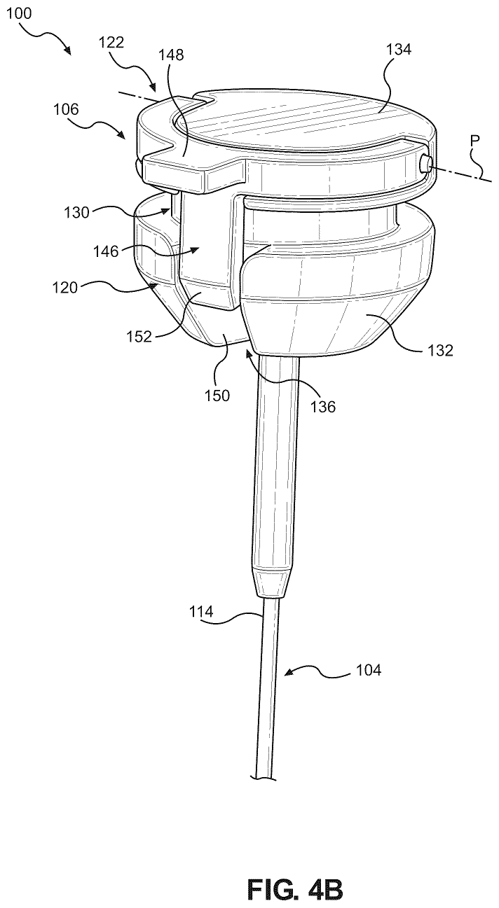

Strand securing device 106 may generally have a base portion 120 and a handle portion 122 moveably coupled to base portion 120 between a first position (FIG. 4A) and a second position (FIGS. 2 and 4B). In an aspect, handle portion 122 is pivotally coupled to base portion 120 such that handle portion 122 pivots between the first and second positions with respect to base portion 120. For example, handle portion 122 can pivot with respect to base portion 120 about a pivot axis P, as seen in FIGS. 4A and 4B. When handle portion 122 is in the first position, base portion 120 may be configured to receive the free ends 12 of the flexible strands 10, and when handle portion 122 is in the second position, the free ends 12 of the flexible strands 10 may be retained and secured between base portion 120 and handle portion 122. As seen in FIG. 4A, when handle portion 122 is in the first position, handle portion 122 may extend in a substantially longitudinal direction with respect to a longitudinal axis L of shaft 104. As seen in FIG. 4B, when handle portion 122 is in the second position, handle portion 122 may extend in a substantially transverse or perpendicular direction with respect to the longitudinal axis L of shaft 104. In another example (not shown), handle portion 122 can be designed as a slider that moves transaxially with respect to base position 120, such as by moving along the pivot axis P or another axis that extends along a second part 134 of base portion 120.

Base portion 120 may comprise a receiving area 130 for assisting with the management of the free ends 12 of the flexible strands 10. Receiving area 130 may be configured to receive the free ends 12 of the flexible strands 10, such that the free ends 12 can be wrapped around base portion 120, as best seen in FIGS. 2 and 3. In an aspect, receiving area 130 may be an annular groove extending substantially around base portion 120, as seen in FIG. 4A. Receiving area 130 generally divides base portion 120 into first and second parts 132 and 134. The first part 132 may be configured to couple to shaft 104 and the second part 134 may be configured to couple to handle portion 122. In an aspect, the first part 132 may comprise a distal attachment 133 that couples to the proximal end 110 of shaft 104, such as by inserting the proximal end 110 of shaft 104 into the distal attachment 133. In an aspect, handle portion 122 is coupled to the second part 134 of base portion 120 such that handle portion 122 can pivot between the first and second positions.

Base portion 120 may comprise a base engagement feature 136, as seen in FIGS. 2, 4A, and 4B. Base engagement feature 136 may be located in the first part 132 of base portion 120. Base portion 120 may also comprise at least one flange 138a. Flange 138a may be positioned on the second part 134 of base portion and can be configured to cooperate with handle portion 122. Another flange 138b may also be provided on the second part 134 of base portion 120 spaced from flange 138a and that can also cooperate with handle portion 122. For example, when handle portion 122 is in the second position, as seen in FIG. 4B, handle portion 122 can rest on flanges 138a and 138b such that flanges 138a and 138b support handle portion 122.

Handle portion 122 may be configured to assist the surgeon with manipulation of inserter 100 when operating by providing easy gripping. Handle portion 122 may include a gripping feature 140 configured to facilitate gripping of handle portion 122. Gripping feature 140 may comprise, for example, a half-ring 142 that defines an open area 144 for gripping the half-ring 142. Ends 145 of half-ring 142 can be pivotally connected to the second part 134 of base portion 120. Open area 144 can be sized to receive one or more fingers of the surgeon. The surgeon need only insert one or more of their fingers into open area 144 of gripping feature 140 to grip handle portion 122 and manipulate inserter 100, such as pulling inserter 100 to remove inserter 100 from the repair site.

Handle portion 122 may include a handle engagement feature 146 configured to releasably engage base engagement feature 136 when handle portion 122 is in the second position, as seen in FIG. 4B. Base and handle engagement features 136 and 146 engage one another in order to retain the free ends 12 of the flexible strands 10 on strand securing device 106, to assist with management of the free ends 12 of the flexible strands 10, such as during the repair procedure. The engagement of base and handle engagement features 136 and 146 can assist with management of the free ends 12 of the flexible strands 10 that are wrapped around base portion 120 at receiving area 130 by keeping the flexible strands 10 neat and secure in receiving area 130 during the repair procedure.

Handle portion 122 may have an actuator 148 for moving handle portion 122 between the first and second positions with respect to base portion 120. In an aspect, actuator 148 may be a tab or projection that extends outwardly from handle portion 122, as seen in FIG. 4A. Actuator 148 may extend from half-ring 142, for example, such as between ends 145 of half-ring 142 adjacent to handle engagement feature 146.

Base and handle engagement features 136 and 146 can form any type of releasably engagement, such as a frictional engagement, a snapping engagement, and the like. For example, base engagement feature 136 may be a generally longitudinal slot 150 that is located at the side 116 of base portion 120, and handle engagement feature 146 may be a projection 152 extending from gripping feature 140 of handle portion 122. Slot 150 may have a wedge shape, for example, such that engagement with a width of projection 152 creates a friction fit. Slot 150 can lead into receiving area 130, thereby allowing the free ends 12 of the flexible strand 10 to extend through slot 150 and into receiving area 130 where the free ends 12 can be wrapped around base portion 120. The releasable engagement between base and handle engagement features 136 and 146 allows the surgeon to easily release the free ends 12 of the flexible strands 10 from strand securing device 106 when needed, such as when insertion of implant 102 is complete and inserter 100 is ready for removal from the repair site. When handle portion 122 is in the second position and base and handle engagement features 136 and 146 are in engagement (e.g. projection 152 is received in slot 150), the surgeon can grab actuator 148 of handle portion 122 to move handle portion 122 to the first position, thereby releasing the engagement between base and handle engagement features 136 and 146 (e.g. removing projection 152 from slot 150). This disengagement of base and handle engagement features 136 and 146 in turn releases the free ends 12 of the flexible strands 10 that were trapped between base and handle engagement features 136 and 146 (e.g. trapped in slot 150 and under projection 152 as seen in FIG. 2). In an aspect, base and handle engagement features 136 and 146 are configured such that the release of the engagement between base and handle engagement features 136 and 146 substantially simultaneously releases the free ends 12 of the flexible strands 10.

An optional second slot 154 (FIG. 3) can be provided on base portion 120 to further assist with management of the flexible strands 10. Second slot 154 can be positioned on the first part 132 of base portion 120 at side 118 thereof (opposite side 116 where slot 150 is located). Second slot 154 can be sized to receive the free ends 12 of the flexible strands 10, as seen in FIG. 3. Second slot 154, like slot 150, can lead into receiving area 130 such that the free ends 12 of the flexible strands 10 can extend through second slot 154 and into receiving area 130. As such, some of the free ends 12 of the flexible strands 10 can extend through second slot 154 in addition to slot 150. This may assist with organizing and distinguishing the flexible strands for the surgeon.

Inserter 100 can be preloaded with implant 102 at tip 113 and the flexible strands 10 coupled to implant 102 and the free ends 12 thereof wrapped around strand securing device 106. Handle portion 122 can be moved to the second position such that base and handle engagement features 136 and 146 are engaged and strand securing device 106 thus acts to fix the flexible strands 10 so that implant 102 stays on tip 113 of inserter 100 and the free ends 12 thereof stay wrapped neatly at strand securing device 106. After insertion of implant 102, handle portion 122 can be raised to expose the free ends 12 of the flexible strands 10 and free the wrapped flexible strands 10.

The design of strand securing device 106 of inserter 100 is beneficial in that it allows the surgeon to put a finger through handle portion 122 for positive control of pulling out inserter 100. Another benefit is that by deploying the strand securing device 106, the free ends 12 of the flexible strands 10 wrapped around base portion 120 can be released substantially simultaneously actuating handle portion 122. This contributes to ease of use and reduction of surgical errors.

As used in this specification and the appended claims, the singular forms “a”, “an” and “the” include plural referents, unless the context clearly dictates otherwise. Similarly, the adjective “another,” when used to introduce an element, is intended to mean one or more elements. The terms “comprising,” “including,” “having” and similar terms are intended to be inclusive such that there may be additional elements other than the listed elements.

It is noted that the description and claims may use geometric or relational terms, such as right, left, above, below, upper, lower, top, bottom, linear, arcuate, elongated, parallel, perpendicular, longitudinal etc. These terms are not intended to limit the disclosure and, in general, are used for convenience to facilitate the description based on the examples shown in the figures. In addition, the geometric or relational terms may not be exact. For instance, walls may not be exactly perpendicular or parallel to one another because of, for example, roughness of surfaces, tolerances allowed in manufacturing, etc., but may still be considered to be perpendicular or parallel.

What is claimed is:

1. An inserter for tissue repair, the inserter comprising:
a shaft having a proximal end and a distal end, the distal end being configured to operatively engage an implant, the implant being coupled to at least one flexible strand; and
a strand securing device located at the proximal end of the shaft, the strand securing device having a base portion and a handle portion moveably coupled to the base portion between a first position and a second position,
wherein, when the handle portion is in the first position, the base portion is configured to receive free ends of the at least one flexible strand, and
wherein, when the handle portion is in the second position, the free ends of the at least one flexible strand are retained between the base portion and the handle portion.

2. The inserter of claim 1, wherein the handle portion is pivotably coupled to the base portion such that the handle portion pivots between the first and second positions with respect to the base portion.

3. The inserter of claim 1, wherein the base portion comprises a receiving area configured to receive the free ends of the at least one flexible strand, such that the free ends of the at least one flexible strand are wrapped around the base portion.

4. The inserter of claim 3, wherein the receiving area is an annular groove extending substantially around the base portion.

5. The inserter of claim 1, wherein the strand securing device is located at a most proximal end of the inserter.

6. The inserter of claim 1, wherein when the handle portion is in the first position, the handle portion extends in a substantially longitudinal direction with respect to a longitudinal axis of the shaft, and when the handle portion is in the second position, the handle portion extends in a substantially transverse direction with respect to the longitudinal axis of the shaft.

7. The inserter of claim 1, wherein the base portion includes a base engagement feature configured to receive the free ends of the at least one flexible strand.

8. The inserter of claim 7, wherein the handle portion includes a handle engagement feature configured to releasably engage the base engagement feature when the handle portion is in the second position.

9. The inserter of claim 8, wherein the base engagement feature is a longitudinal slot and the handle engagement feature is a projection extending from the handle portion.

10. The inserter of claim 8, wherein the base engagement feature and the handle engagement feature frictionally engage.

11. The inserter of claim 1, wherein the handle portion includes a gripping feature configured to facilitate gripping of the handle portion.

12. The inserter of claim 1, wherein the handle portion includes an actuator for moving the handle portion between the first and second positions.

13. The inserter of claim 12, wherein the actuator is a tab extending outwardly from the handle portion.

14. The inserter of claim 1, wherein the base portion includes at least one flange configured to support the handle portion when the handle portion is in the second position.

15. An inserter for tissue repair, the inserter comprising:
a shaft having a proximal end and a distal end, the distal end being configured to operatively engage an implant, the implant being coupled to at least one flexible strand; and
a strand securing device located at the proximal end of the shaft, the strand securing device having a base portion and a handle portion moveably coupled to the base portion between a first position and a second position, the base portion comprises a receiving area for receiving free ends of the at least one flexible strand,
wherein the base portion has a base engagement feature and the handle portion has a handle engagement feature to releasably retain the free ends of the at least one flexible strand between the base portion and the handle portion when the handle portion is in the second position.

16. The inserter of claim 15, wherein the base portion includes at least one flange configured to support the handle portion when the handle portion is in the second position.

17. The inserter of claim 15, wherein the base and handle engagement features engage in a frictional engagement when the handle portion is in the second position.

18. The inserter of claim 15, wherein the handle portion includes an actuator for moving the handle portion between the first and second positions.

19. The inserter of claim 15, wherein the handle portion is pivotably coupled to the base portion such that the handle portion pivots between the first and second positions with respect to the base portion.

20. The inserter of claim 15, wherein the receiving area is an annular groove extending substantially around the base portion.
